(19) United States
(12) Patent Application Publication
(10) Pub. No.: US 2014/0039243 A1
(43) Pub. Date: Feb. 6, 2014

(54) MINIMALLY INVASIVE SLINGS FOR FEMALE URINARY STRESS INCONTINENCE

(71) Applicants: Pier Aldo Crepaldi, Livorno Ferraris (IT); Roberta Lamberti, Ulzio (Torino) (IT); Donato Piroli Torelli, Napoli (IT); Ermanno Trabucco, Muttontown, NY (US)

(72) Inventors: Pier Aldo Crepaldi, Livorno Ferraris (IT); Roberta Lamberti, Ulzio (Torino) (IT); Donato Piroli Torelli, Napoli (IT); Ermanno Trabucco, Muttontown, NY (US)

(73) Assignee: Hemiamesh S.r.l., Chivasso (Torino) (IT)

(21) Appl. No.: 13/641,889

(22) PCT Filed: Apr. 19, 2011
(86) PCT No.: PCT/IB11/51686

§ 371 Date: Nov. 16, 2012

(30) Foreign Application Priority Data
Apr. 19, 2010 (IT) TO2010A000314

(51) Int. Cl. A61F 2/00

(52) U.S. Cl. CPC A61F 2/0045
USPC 600/30

(57) ABSTRACT

Minimally invasive slings for the treatment and prevention of female urinary stress incontinence and related disorders are provided. Such slings include those having a first mesh of synthetic, biocompatible, non-absorbable polymer which extends from one end portion to the other through the median portion and a second mesh of synthetic, biocompatible, non-absorbable polymer having average porosity lower than the average porosity of the first mesh.

The present invention relates to a device for treating urinary incontinence, a common disorder which predominantly affects women. It has been estimated that more than 13 million individuals suffer from urinary incontinence in the United States, 85% of them women. Regarding the prevalence of urinary incontinence in women from four different European countries (France, Germany, Spain and the UK), 35% of women interviewed who wished to provide an answer reported involuntary losses of urine in the 30 days preceding the interview. The type of incontinence was predominantly stress incontinence (S. Hunskaar et al—The prevalence of urinary incontinence in women in four European countries—BJU International, Volume 93 Issue 3, Pages 324-330, published online: 4 Feb. 2004). Type I and II stress incontinence is caused by urethral hypermobility, a condition in which the pelvic floor is weakened or damaged, leading to the lowering of the neck of the bladder and/or the proximal urethra in response to an increase in intra-abdominal pressure. This pressure may be due to various routine daily activities such as laughing, sneezing, coughing, lifting weights, walking or getting up from a seat. The result is an inadequate sphincter response of the urethra and a consequent loss of urine. Biological factors which may cause hypermobility include insufficient endopelvic muscle tone (due to age or limited physical activity), stretching caused by injury to the endopelvic fascia, due to pregnancy for example, separation of muscle and ligament (fascia and arcus tendineus), or hormone (oestrogen) deficiency.

In order to increase the urethral closure pressure and thus mitigate involuntary loss of urine, a support is provided under the urethra by a surgical procedure.

The surgical treatment most commonly used at present for female urinary stress incontinence requires the implantation of what is known as a “sling” in a sub-urethral position. Conventionally, slings are inserted under the urethra in order to provide a bearing and support surface to limit the prolapse of the endopelvic muscles as far as possible at the time when the urethral sphincter is subject to compression. The complexity of the surgical procedure and the technical difficulties regarding the anatomical positioning of the sling have caused problems for surgeons and patients for some time. Some procedures for implanting urethral supports require multiple incisions in the patient, and, in some cases, the sling is also tensioned from outside the patient's body after the operation.

For many other commonly implanted slings, however, fixing or anchoring to bone, tissues, skin or muscles by screwing or suturing is required. In fact, there is no need to provide any particular means of tensioning or fixing the sling to achieve continence, because the urethra is not stabilized and supported by the sling itself, but rather by the reaction of the tissue fibres which develop through the pores of the sling. The process of fibrosis promotes the adhesion of the sling to the adjacent tissues, and this is the basis of the suspension mechanism used in the tension-free procedure (D. Piroli Torelli et al.—SUS for the correction of stress urinary incontinence: towards more and more simplified surgery—Urogynaecologia International Journal 2008; 22; 8: 5-15).

Indeed, the tension-free TVT (trans-vaginal tape) or TOT (trans-obturator tape) surgical procedures are most commonly used, although even in these cases multiple incisions are required and insertion needles have to be used for the insertion of the device. The sling can normally be up to 45 cm long: consequently the excess material not directly concerned with urethral support extends through the abdominal wall, in the case of the TVT method, or the obturator foramen, in the case of the TOT method, and remains permanently implanted as a possible source of infection and discomfort throughout the patient's lifetime.

Technical problems and serious complications can arise during the passage of the needles. For example, some typical complications of the TVT method include possible perforation of the bladder, requiring cystoscopy, perforations of the intestine, nerve and blood vessel damage which may cause intra-operative bleeding, and death (Daniel Rapoport, MD, Howard N. Fenster, MD, Jamie E. Wright, MD—Reported complications of tension-free vaginal tape procedures: A review—BCMJ, Vol. 49, No. 9, November 2007, page(s) 465-524).

The TOT method too can give rise to potential complications: these include occasional intra-operative haemorrhages, pain and discomfort in the patient, infections, bleeding and post-operative urethral obstruction (Neuman M.—TVT-Obturator: Short-term data on an operative procedure for the cure of female stress urinary incontinence performed on 300 patients—Eur Urol 2007; 51: 1083-1087; Rezapour M, Novara G, Meier P A et al.—A three-month pre-clinical trial to assess the performance of a new TVT-like mesh (TVTx) in a sheep model—Int Urogynecol J Pelvic Floor Dysfunc 2007; 18: 183-187). The pain which develops in the inguinal and hip area is a consequence of the lithotomy position with the legs wide apart which the patient has to assume during the operation in order to provide better access to the obturator foramen; this complication becomes more serious in the case of women with coxarthrosis or reduced joint function.

The prior document WO2008/067 317 describes a sling comprising an oblong median portion and end portions for anchoring to the tissues, in which the sling is implanted surgically. This median portion is made from biocompatible polymer mesh and is associated with bioabsorbable polymer end portions having saw-tooth or pointed protrusions. The end portions are rather bulky and consequently invasive in relation to the tissues in which they are to be implanted, and are therefore made from bioabsorbable polymer in such a way that they can disappear over time.

Another prior document, WO2005/122 954, describes a sling formed by a mesh folded back on itself at the ends to form corresponding pockets for the engagement of instruments for positioning the device which do not perforate the abdominal wall.

US-2005/0267325 describes generic implantable surgical articles, and is mainly concerned with methods of joining different materials or layers of the same material. Drawings in US-2005/0267325 illustrate a sling comprising a first mesh having end parts whose two faces are covered by a corresponding second mesh which is Y-shaped, the branches of the Y being adjacent to the corresponding faces of the associated end part of the first mesh, and the stem of the Y forming the corresponding end portion of the sling. Thus the first mesh does not extend from one end portion of the sling to the other. The two parts of the Y-shaped second mesh are joined to the first mesh by a corresponding polymer rivet passing through an aperture perpendicular to the general plane of the sling, and in order to achieve this it is typically necessary to carry out an ad hoc process which may promote the formation of gaps in the meshes. It should be noted that this structure with three layers joined by a rivet, which is the essence of what is described in US-2005/0267325, makes the ends of the sling rigid and bulky.

US-2002/0028980 describes an implantable article comprising a base portion, from one end of which two engagement portions extend independently of each other. This article has a Y-shaped profile, in other words a profile which is not compact. Additionally, US-2002/0028980 simply asserts that the article described therein can comprise, in a non-limiting way, pores having dimensions ranging from 1.016 to 1.397 mm, without mentioning the average dimension or the possibility of additionally having pores of different dimensions, but solely stating that the choice of pore dimensions is dictated by considerations relating to the fixing to the surrounding tissues.

The object of the present invention is therefore to provide a device which is improved with respect to the prior art, and which, in particular, is free of the aforementioned disadvantages and can be used for simpler and safer surgical procedures.

According to the invention, this object is achieved by means of a sling comprising an oblong median portion and anchoring end portions, with a first mesh of synthetic, biocompatible, non-absorbable polymer extending from one end portion to the other through the median portion, and a second mesh of synthetic, biocompatible, non-absorbable polymer having average porosity lower than the average porosity of the first mesh, but nevertheless greater than 100 μm, being joined to and superimposed on the parts of the first mesh belonging to the end portions.

In the present description, the term “average porosity of a mesh” denotes, in particular, the arithmetic mean of the dimensions of the pores present between the filaments, which is determined by the conditions and by the type of weaving.

The sling according to the invention therefore combines a first mesh (such as that described in Italian patent application MI2009A001186, the content of which is incorporated herein by reference) having high porosity, reduced elasticity and a high axial breaking load, with a second mesh which stiffens the end portions which still remain flexible and macroporous to some extent. Macroporosity is a key attribute of the sling according to the invention, because it not only promotes fibroblast infiltration which is the principle on which strong and permanent anchorage is based, but also minimizes the onset of any infection. This is because bacteria can invade all surgical meshes because their average diameter is about 1 μm, but if the pores of the mesh are large enough to allow the infiltration of macrophages, which are immune cells with dimensions of about 10 μm, responsible for the phagocytosis of bacteria, then defence against infection can take place. Since the average porosity of the end portions of the sling according to the invention is greater than 100 μm, as mentioned above, the sling has the advantage of permitting both macrophage and fibroblast infiltration.

Anchoring in the immediate post-operative period takes place by means of the stiffened end portions which provide a strong attachment in the periurethral connective tissue. Another factor helping to keep the sling in position is that it is subject to two opposing pressures, one from the outside to the inside, created by the vaginal mucosa and the underlying fascia, and the other from the inside to the outside, created by the intra-abdominal pressure, both acting within an anatomically closed space. The positioning of the sling, which is not attached in a fixed position to the obturator membrane, can thus be described actually as “tension free”. A few hours after the operation, the pores of the sling are infiltrated by fibrous tissue neoformations which also assist with the anchorage.

Because of its specific structure, the overall length of the sling according to the invention can be shorter than that of similar known devices, whose length is such that the membrane is perforated. Conversely, the length of the sling according to the invention is about 66 mm, thus permitting pre-obturator positioning which follows the same direction as the aforementioned TOT slings of the prior art.

Considered as a whole, the sling according to the invention represents a markedly smaller quantity of foreign matter for the body in which it is inserted, but it can still be implanted in an adequate and permanent way.

Furthermore, the sling according to the invention enables the advantageous “single incision” operating procedure to be used, thus reducing the passage of surgical instruments to a minimum while also limiting intra-operative risks and post-operative pain.

Other advantages and characteristics of the present invention will become clear from the following detailed description which is given by way of non-limiting example with reference to the attached drawing, in which:

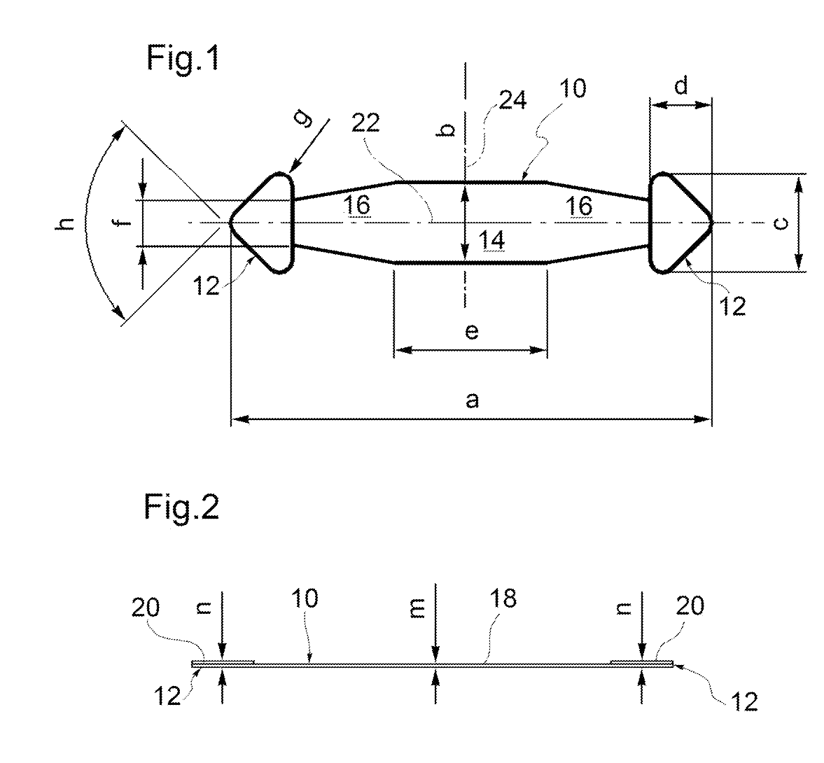

FIG. 1 is a plan view of the sling according to the invention, and

FIG. 2 is a view in side elevation of the sling of FIG. 1.

A sling for supporting the urethra comprises an oblong median portion 10 and anchoring end portions 12. For the purpose of definition, and as clearly shown in the drawings, the end portions 12 necessarily include the areas of the sling farthest from the centre.

The median portion has a shape which is generally tapering from the centre towards the ends, and in particular it is formed by a substantially rectangular central part 14 with a perimeter formed by a pair of short sides and a pair of long sides, and by end parts 16 of substantially trapezoidal shape, the longer bases of which correspond to the short sides of the central part.

Each end portion 12 is substantially arrow-shaped with a tip facing outwards, the width at the base of each arrow being greater than the maximum width of the median portion 10.

A first mesh 18 extends from one end portion 12 to the other through the whole median portion 10. A corresponding second mesh 20 having an average porosity smaller than that of the first mesh 18 is superimposed on the whole extension of the parts of the first mesh 18 belonging to the end portions 12. The two meshes 18 and 20 are joined to each other continuously over the whole contact surface, for example by welding or stitching or by any other method which does not obstruct their pores, preferably by ultrasonic welding.

Thus the meshes 18 and 20 are joined without the need for any additional bulky elements such as rivets or the like, which would stiffen the sling and make it unwieldy, and in this way the sling is provided with a compact configuration.

It should also be noted that, as FIG. 2 clearly shows, the second mesh 20 covers only one face of each of the end portions of the first mesh 18 which coincide with the end portions 12 of the sling as a whole, while the opposite faces of the end portions of the first mesh 18 are left free.

Considered as a whole, the sling is symmetrical about a longitudinal axis 22 and also about a transverse axis 24 lying in its general plane, and is constituted by the meshes 18 and 20.

Advantageously, the first mesh 18 has a nominal density in the range from 30 to 60 g/m², preferably 48 g/m², and an average porosity in the range from 800 μm to 1200 μm, preferably 1000 μm, while the second mesh 20 has a nominal density in the range from 100 to 160 g/m², preferably 127 g/m², and an average porosity in the range from 500 μm to 900 μm, preferably 700 μm.

The first and second meshes 18 and 20 can be formed independently from filaments of any non-absorbable biocompatible synthetic polymer, for example a polymer chosen from the group composed of homopolymers and copolymers of polypropylene, polyethylene, polyester, polyamide, partially or totally fluorinated polymers and mixtures thereof. The filaments of the meshes 18 and 20 can also be covered with any biocompatible material (whether bioreabsorbable or not), provided that the aforementioned porosity parameters are adhered to. In particular, the mesh 18 can be made from 80 μm PP monofilament, and the mesh 20 can be made from 180 μm PP monofilament.

By way of example, the sling may have an overall length “a” in the range from 55 to 75 mm, preferably 66 mm, while each end portion 12 can have a length “d” in the range from 5 to 15 mm, preferably 8.5 mm.

Also by way of example, the shorter base of each end part 16 of the median portion 10 can have a length “f” in the range from 5 to 9 mm, preferably 6.5 mm, while each long side can have a length “e” in the range from 10 to 40 mm, preferably 21 mm, and each short side can have a length “b” in the range from 9 to 12 mm, preferably 11 mm.

Advantageously, each arrow-shaped end portion 12 has a width at the base “c” in the range from 11 to 16 mm, preferably 13.5 mm, and a vertex angle “h” in the range from 75° to 105°, preferably 90°. Considered as a whole, each arrow is shaped in the form of an isosceles triangle with rounded angles “g”.

In preferred embodiments, the thickness “m” of the median portion 10 is in the range from 0.25 to 0.35 mm, preferably 0.3 mm, while the thickness “n” of the end portions 12 due to the superimposition of the meshes 18 and 20 is in the range from 0.4 to 0.6 mm, preferably 0.5 mm.

For the surgical implantation of a sling of the type described above, a suburethral vaginal incision is initially made with a length of about 1 cm, after which two tunnels with a length of about 3.5 cm are made to the right and left of the urethra, following the path used in the TOT method, until contact is made with the obturator membranes without perforating them. One of the arrow-shaped end portions 12 is then folded back on itself and is grasped with a curved Klemmer Mosquito forceps so that it can be inserted into the first transobturator tunnel The same operations are then carried out on the other end portion 12 to insert it into the second tunnel.

Finally, a colporrhaphy is performed on the vaginal incision, with one or two separate introflecting stitches.

Naturally, the principle of the invention remaining the same, the details of construction and the forms of embodiment may be varied widely with respect to those described, which have been given purely by way of example, without thereby departing from the scope of the invention as defined in the attached claims.

1-15. (canceled)

16. A sling for supporting the urethra, comprising an oblong median portion and anchoring end portions, wherein a first mesh of synthetic, biocompatible, non-absorbable polymer extends from one end portion to the other through the median portion, a second mesh comprising synthetic, biocompatible, non-absorbable polymer having an average porosity lower than the average porosity of the first mesh, but greater than 100 μm, being joined to and superimposed on the end portions of the first mesh.

17. The sling of claim 16, wherein said first mesh has a nominal density from about 30 to about 60 g/m² and an average porosity from about 800 to about 1200 μm.

18. The sling of claim 17, wherein said second mesh has a nominal from about 100 to about 160 g/m² and an average porosity from about 500 to about 900 μm.

19. The sling of claim 16, wherein the thickness of the median portion is from about 0.25 to about 0.35 mm, and the thickness of the end portions is from about 0.4 to about 0.6 mm.

20. The sling of claim 16, wherein said second mesh is joined to the first mesh over the whole extension of the end portions.

21. The sling of claim 16, having an overall length from about 55 to about 75 mm, each end portion having a length from about 5 to about 15 mm.

22. The sling of claim 16, wherein said median portion has a generally tapering shape from the centre towards the ends.

23. The sling of claim 22, wherein said median portion is formed by a substantially rectangular central part having a perimeter formed by a pair of shorter sides and a pair of longer sides, and by end parts having a substantially trapezoidal shape, in which the longer bases correspond to the shorter sides of the central part.

24. The sling of claim 23, wherein the shorter base of each end part of the median portion has a length from about 5 to about 9 mm.

25. The sling of claim 23, wherein each longer side has a length from about 10 to about 40 mm, and wherein each shorter side has a length from about 9 to about 12 mm.

26. The sling of claim 16, wherein each end portion is substantially arrow-shaped with a tip facing outwards, the width at the base of each arrow being greater than the maximum width of the median portion.

27. The sling of claim 26, wherein each arrow has a width at the base from about 11 to about 16 mm and a vertex angle in a range from about 75° to about 105°.

28. The sling of claim 16, wherein said first and second meshes are independently formed from filaments of polymer chosen from the group consisting of: homopolymers and copolymers of polypropylene, polyethylene, polyester, polyamide, partially or totally fluorinated polymers and any combination thereof.

29. The sling of claim 28, wherein said filaments comprise a coating of biocompatible material.

30. The sling of claim 16, wherein said second mesh covers only one face of each of the end portions of said first mesh, and the opposite face is left free.
